(19) United States
(12) Plant Patent Application Publication
(10) Pub. No.: US 2006/0041972 P1
(43) Pub. Date: Feb. 23, 2006

(54) Double impatiens plant named 'Cameo Orange'

(75) Inventor: Xenia Cascante, Alajuela (CR)

Correspondence Address: THE WEBB LAW FIRM, P.C., 700 KOPPERS BUILDING, 436 SEVENTH AVENUE, PITTSBURGH, PA, 15219, US

(73) Assignee: Oglevee, Ltd., Connellsville, PA (US)

(21) Appl. No.: 11/209,338

(22) Filed: Aug. 23, 2005

Related U.S. Application Data
(60) Provisional application No. 60/603,849, filed on Aug. 23, 2004.

(51) Int. Cl. A01H 5/00

(52) U.S. Cl. PLT/317

(57) ABSTRACT

A new and distinct cultivar of Double Impatiens plant named ‘Cameo Orange’, characterized by its compact, mounded and outwardly spreading plant habit; rapid growth rate; freely branching habit; dense foliage; freely flowering habit; fully double flower form; and large orange flowers that are positioned above and beyond the foliage.

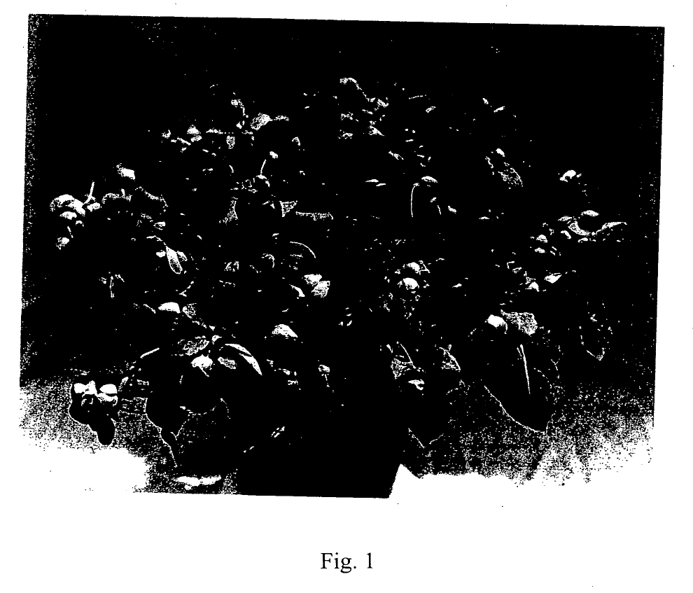

### CROSS-REFERENCE TO RELATED APPLICATIONS

This application claims the benefit of U.S. Provisional Plant Patent Application No. 60/603,849 filed Aug. 23, 2004, which is hereby incorporated by reference in its entirety.

### BOTANICAL CLASSIFICATION

Impatiens walleriana

### VARIETAL DENOMINATION

‘Cameo Orange’

### BACKGROUND OF INVENTION

The present invention comprises a new and distinct cultivar of Impatiens walleriana, and known by the varietal name ‘Cameo Orange’. The new variety is a product of a planned breeding program conducted in Cartago, Costa Rica. The purpose of the breeding program was to develop compact and mounded cultivars with fully double flowers, large flower size, and interesting flower colors. The new variety originated from a cross of two proprietary selections of Impatiens walleriana, not patented. Comparisons between the new variety and its parents cannot be made, as the parental varieties no longer exist. The cultivar ‘Cameo Orange’ was discovered and selected as a flowering plant within the progeny of the stated cross in controlled environment in Cartago, Costa Rica. Asexual reproduction of the new cultivar by terminal cuttings has shown that the unique features of the new variety are stable and reproduced true to type in successive propagations.

The following traits have been repeatedly observed and are determined to be the unique characteristics of ‘Cameo Orange’. Characteristics that distinguish ‘Cameo Orange’ as a new and distinct cultivar from others known to the breeder include:

1) Large flower size of 4.5 to 5.0 cm in diameter;

2) Compact, mounded, spreading growth habit;

3) Freely branching habit;

4) Rounded leaves with dense foliage;

5) Time to produce a finished plant in a 10 cm pot is 5 to 6 weeks; and

6) Orange flowers are carried above the leaf canopy and face upward.

Plants of the new variety can be compared to plants of the Impatiens walleriana cultivar ‘Fiesta Deep Orange’. In side-by-side comparisons conducted in Lompoc, Calif., plants of the new variety differed from plants of the cultivar ‘Fiesta Deep Orange’ in the following characteristics:

1) Plants of the new variety had peduncles of a color of 146B, whereas the plants of ‘Fiesta Deep Orange’ had peduncles of a color of 147C;

2) Plants of the new variety had flowers with a color of 41A, whereas plants of ‘Fiesta Deep Orange’ had flowers with a color of 44D with streaks of 41D;

3) Plants of the new variety had a stem color of 146D with a cast of 184C, whereas plants of ‘Fiesta Deep Orange’ had a stem color of 148C with cast of 184C mostly around the nodes;

4) Plants of the new variety had mature leaves that were longer and wider leaves than plants of ‘Fiesta Deep Orange’;

5) Plants of the new variety were taller than plants of ‘Fiesta Deep Orange’; and

6) Plants of the new variety had a mounded and spreading plant habit, whereas the plants of ‘Fiesta Deep Orange’ had a mounded to semi-upright plant habit.

Plants of the new varaiety can also be compared to plants of the Impatiens walleriana cultivar ‘Fiesta Orange Spice’. In side-by-side comparisons conducted in Lompoc, Calif., plants of the new variety differed from plants of ‘Fiesta Orange Spice’ in the following characteristics:

1) Plants of the new variety had a mounded spreading plant habit, whereas plants of ‘Fiesta Orange Spice’ had a semi-upright plant habit;

2) Plants of the new variety had leaves that were longer and wider than plants of ‘Fiesta Orange Spice’;

3) Plants of the new variety had a stem color of 146D with a cast of 184C, whereas plants of ‘Fiesta Orange Spice’ had a stem color of 147C with flecks of 184C;

4) Plants of the new variety had flowers with a color of 41A, whereas plants of ‘Fiesta Orange Spice’ had flowers with a color of 33B; and

5) Plants of the new variety had peduncles with a color of 146B, whereas plants of ‘Fiesta Orange Spice’ had peduncles with a color of 148C.

### BRIEF DESCRIPTION OF THE DRAWING

The accompanying colored photographic drawing illustrates the new variety with the colors being as nearly true as is possible to obtain in colored illustrations of this type.

### DESCRIPTION OF THE PLANT

The cultivar ‘Cameo Orange’ has not been observed under all possible environmental conditions. The phenotype may vary somewhat with variations in environment such as temperature, day length and light intensity. However there is no variance in genotype.

In the following description, color references are made to The Royal Horticultural Society Colour Chart, 1995 Edition, except where general terms of ordinary dictionary significance are used. Plants were grown under commercial practice during the winter of 2003 in Lompoc, Calif., in a corrugated polycarbonate-covered greenhouse with 30% shade covering. During the production of the plants, day temperatures ranged from 21 to 24° C.; night temperatures ranged from 16 to 18° C.; and light levels ranged from 4000 to 5000 foot candles of light. Plants used in the following description were grown in 15 cm containers for about nine to ten weeks after planting rooted cuttings.
Propagation:
   Type cutting.—Terminal cuttings.
   Time to initiate roots.—Summer: About 10 days at 21° C. Winter: About 12 days at 21° C.
   Time to produce a rooted cutting.—Summer: About 21 days at 21° C. Winter: About 21 days at 21° C.
   Root description.—Fibrous, fine, white in color.
   Root habit.—Freely branching, dense.
Plant description:
   General appearance.—Compact, mounded and outwardly spreading. Appropriate for 10-15 cm pots and 20 to 25 cm hanging basket containers.
   Growth and branching habit.—Vigorous and freely-branching habit with at least 10 lateral branches developing per plant, dense growth. Pinching (removal of terminal apex) is usually not required.
   Crop time.—Relatively rapid growth rate. From planting one rooted cutting in a 10 cm pot: About 5 to 6 weeks. From planting three rooted cuttings in a 25 cm hanging basket container: About 8 to 10 weeks.
   Plant height at flowering.—About 18 to 20 cm.
   Plant width (spread).—About 28 cm.
   Lateral branch description.—Length: About 15 to 20 cm. Diameter: About 8 to 10 mm. Internode length: About 1 to 2 cm. Texture: Smooth, glabrous. Color: 146D with a cast of 184C.
   Foliage description.—Arrangement: Alternate, simple; densely foliated. Length: About 5 to 5.5 cm. Width: About 2.5 to 3 cm. Shape: Ovate. Apex: Acute. Base: Acute to rounded. Margin: Slightly serrated with fine ciliation. Texture, upper and lower surfaces: Glabrous. Venation pattern: Pinnate. Color: Developing leaves, upper surface: 147A. Developing leaves, lower surface: 148A. Fully expanded leaves, upper surface: 146A. Fully expanded leaves, lower surface: 146C with cast of 184D in some areas. Venation, upper surface: 146C. Venation, lower surface: 146B. Petiole length: About 10 to 12 mm. Petiole diameter: About 3 mm. Petiole texture, upper and lower surface: Smooth, glabrous. Petiole color: 146C with a slight cast of 184D.
Flower description:
   Flower type and habit.—Numerous and consistently double orange flowers. Freely and continuously flowering. Flower buds open similar to a rose in fullness; flowers rounded. Usually 8 to 15 flowers and flower buds per lateral branch. Flowers positioned above the foliage and typically face outward to upward. Flowers last about 7 to 10 days on the plant. Flowers not persistent. Flowers not fragrant.
   Flowering season.—Year-round under greenhouse conditions. In the garden, flowering from spring until fall. Flowering indeterminate and continuous.
   Flower diameter.—About 4.5 to 5 cm.
   Flower depth (height).—About 2 cm.
   Flower buds.—Rate of opening: From full color bud to full open flower, about 5 to 7 days is typical depending on temperature. Length: About 15 mm. Diameter: About 12 mm. Shape: Obovate. Color (just before opening): 41B.
   Petals.—Quantity/arrangement: About 20 to 25; imbricate. Length, outer petals: About 20 to 22 mm. Width, outer petals: About 18 to 20 mm. Shape: Cordate to ovate. Apex: Rounded with most petals showing indentations. Base: Acute. Margin: Entire. Texture, upper and lower surfaces: Glabrous, smooth. Color: When opening, upper surface: 40A. When opening, lower surface: 41C. Fully opened, upper surface: 41A. Fully opened, lower surface: 48B.
   Peduncles.—Length: About 30 to 35 mm. Diameter: About 2 mm. Angle: About 45° to the lateral branch. Strength: Strong. Texture: Smooth, glabrous. Color: 146B.
Reproductive organs: Not applicable due to full double flowering.
Fruit/Seed development: Fruit and seed development has not been observed.
Disease/Pest resistance: Plants of the new Impatiens have not been observed to be resistant to pathogens and pests common to Impatiens.
Temperature tolerance: Plants of the new Impatiens have been observed to be tolerant to rain, wind, and temperatures ranging from 5 to 38° C.

I claim:

1. A new and distinct variety of Impatiens named ‘Cameo Orange’ as herein described and illustrated
